(19) United States
(12) Patent Application Publication
(10) Pub. No.: US 2021/0196500 A1
(43) Pub. Date: Jul. 1, 2021

(54) AUTOMATIC ADJUSTABLE SUPPORTING EQUIPMENT AND METHOD FOR AUTOMATICALLY ADJUSTING SUPPORTING EQUIPMENT

(71) Applicant: Industrial Technology Research Institute, Hsinchu (TW)

(72) Inventors: Guang-Huei Gu, Tainan City (TW); Shih-Ching Huang, Kaohsiung City (TW); Chih-Jen Chen, Tainan City (TW)

(73) Assignee: Industrial Technology Research Institute, Hsinchu (TW)

(21) Appl. No.: 17/136,060

(22) Filed: Dec. 29, 2020

Related U.S. Application Data
(60) Provisional application No. 62/954,664, filed on Dec. 30, 2019.

(30) Foreign Application Priority Data
Nov. 23, 2020 (TW) 109140973

(51) Int. Cl. A61F 5/01; G16H 20/30; G16H 40/63; G01C 19/00; G01P 15/00

(52) U.S. Cl. CPC A61F 5/0123; G16H 20/30; G16H 40/63; A61F 2005/0155; G01P 15/00; A61F 2005/0188; G01C 19/00

(57) ABSTRACT

An automatic adjustable supporting equipment, including a supporting band, an adjusting device, and a controller, is provided. The supporting band wraps around a limb of a user. The adjusting device is disposed on the supporting band, and includes an actuating mechanism and an accelerometer. The actuating mechanism is configured to adjust a pressure applied on the limb by the supporting band. The accelerometer is configured to detect an acceleration value. The controller is coupled to adjusting device, and is configured to drive the actuating mechanism to adjust the pressure to a preset pressure value according to the acceleration value.

### CROSS-REFERENCE TO RELATED APPLICATION

This application claims the priority benefit of U.S. provisional application serial no. 62/954,664, filed on Dec. 30, 2019, and Taiwan application serial no. 109140973, filed on Nov. 23, 2020. The entirety of each of the above-mentioned patent applications is hereby incorporated by reference.

### BACKGROUND

### Technical Field

This disclosure relates to a supporting equipment and a method for adjusting the supporting equipment, and in particular to an automatic adjustable supporting equipment and a method for automatically adjusting the supporting equipment.

### Description of Related Art

In recent years, due to technological advancements, many tasks that used to require manual labor to complete have been largely replaced by mechanical labor. Although this brings a lot of convenience to the lives of people, opportunities for the human body to be active have gradually reduced. Lifestyle of the general population has gradually moved from one that is active to one that is sedentary, which inevitably leads to a gradual decline in physical fitness. In terms of physical fitness, in the past, emphasis was placed on evaluation and improvement of cardiopulmonary function, while other aspects of physical fitness are often neglected. This not only leads to unbalanced improvement of physical fitness, but also reduces the effectiveness of training. Decline of muscle fitness during exercise is one of the most common causes of civilizational diseases, for example, low back pain, which is mostly caused by myogenic problems during exercise such as muscle weakness or muscle tightness.

As a result, the industry has invented a variety of supporting equipment, which are capable of holding a limb of a user in a relatively stable position to ensure that it is not easily injured. However, the supporting equipment created by current technology still has shortcomings. For example, in order to achieve better protection and enhance muscle strength, the supporting equipment should cover the limb as tightly as possible, but this will cause muscle strength of the user to deteriorate and discomfort after a long period of use, making it difficult to balance protection and comfort.

### SUMMARY

According to an embodiment of the present disclosure, an automatic adjustable supporting equipment includes a supporting band, an actuating mechanism, an accelerometer, and a controller. The supporting band wraps around a limb of a user. The actuating mechanism is assembled with the supporting band, and is configured to adjust a pressure applied on the limb by the supporting band. The accelerometer is configured to detect an acceleration value. The controller is coupled to the actuating mechanism and the accelerometer, and is configured to drive the actuating mechanism to adjust the pressure to a preset pressure value according to a change in the acceleration value.

According to an embodiment of the present disclosure, an automatic adjustable supporting equipment includes a supporting band, an actuating mechanism, and a controller. The supporting band is suitable for wrapping around a limb of a user. The actuating mechanism includes a motor and an electromagnetic valve. The motor is suitable for assembling with the supporting band, and is configured to drive the supporting band to adjust a pressure applied on the limb by the supporting band. The electromagnetic valve is configured to switch between an engaged position and a rotation position, and includes a stopping piece. The controller is coupled to the actuating mechanism, and is capable of controlling the electromagnetic valve to switch to the engaged position or the rotation position. The stopping piece of the electromagnetic valve is engaged with an axis of rotation of the motor to block rotation of the motor when the electromagnetic valve is in the engaged position, and the stopping piece of the electromagnetic valve is disengaged from the axis of rotation to enable the motor to rotate freely when the electromagnetic valve is in the rotation position.

According to an embodiment of the present disclosure, a method for automatically adjusting a supporting equipment includes the following steps. A supporting band is used to wrap around a limb of a user. An acceleration value of the limb is detected by the accelerometer. An action state of the user is determined by the controller according to a change in the acceleration value, and the supporting band is adjusted accordingly, so as to enable a pressure applied on the limb by the supporting band to be equal to a preset pressure value, in which the preset pressure value corresponds to the action state.

Based on the above, the automatic adjustable supporting equipment of the disclosure may determine the action state of the user according to the motion parameter of the limb detected by the sensor, and drive the actuating mechanism to adjust (increase or reduce) the pressure applied on the limb by the supporting band according to the action state. Therefore, the actuating mechanism may increase the pressure (tightening) applied on the limb by the supporting band when the user is in the dynamic action state, so as to increase supporting force and restraining force on the limb. The actuating mechanism may reduce the pressure (releasing) applied on the limb by the supporting band to improve the comfort of the user when the user is in the static action state.

### BRIEF DESCRIPTION OF THE DRAWINGS

FIG. 1 is a schematic diagram of an automatic adjustable supporting equipment of an embodiment of the disclosure.

FIG. 1A is a schematic diagram of the automatic adjustable supporting equipment of another embodiment of the disclosure.

FIG. 2 is a schematic block diagram of the automatic adjustable supporting equipment of an embodiment of the disclosure.

FIG. 2A is a schematic block diagram of the automatic adjustable supporting equipment of another embodiment of the disclosure.

FIG. 3 is a schematic diagram of an actuating mechanism of the automatic adjustable supporting equipment of an embodiment of the disclosure.

FIGS. 4 and 5 are schematic diagrams of the actuating mechanism of the automatic adjustable supporting equipment in two statuses according to another embodiment of the disclosure.

FIG. 6 is a schematic block diagram of the automatic adjustable supporting equipment of another embodiment of the disclosure.

FIG. 7 is a schematic flowchart of a method for automatically adjusting the supporting equipment of an embodiment of the disclosure.

FIG. 7A is a schematic flowchart of a method for automatically adjusting the supporting equipment of another embodiment of the disclosure.

FIGS. 8 and 9 are schematic diagrams of the automatic adjustable supporting equipment when used in different action states according to an embodiment of the disclosure.

FIG. 10 is a schematic curve diagram of angles detected by an angle sensor in different action states according to an embodiment of the disclosure.

FIGS. 11 and 12 are schematic diagrams of the automatic adjustable supporting equipment when used in different action states according to an embodiment of the disclosure.

FIG. 13 is a schematic curve diagram of the angles detected by an angle sensor in different action states according to an embodiment of the disclosure.

FIG. 14 is a schematic curve diagram of accelerations detected by an accelerometer in another action state according to an embodiment of the disclosure.

FIG. 15 is a schematic curve diagram of angles detected by the angle sensor in another action state according to an embodiment of the disclosure.

### DESCRIPTION OF THE EMBODIMENTS

The aforementioned and other technical contents, features, and effects of this disclosure will be clearly presented in the following detailed description of each embodiment with reference to drawings. Directional terminology mentioned in the following embodiments, for example, “up”, “down”, “front”, “rear”, “left”, “right”, etc., are used in reference to the orientation of the attached drawings. As such, the directional terminology is used for purposes of illustration and is in no way limiting. In addition, in the following embodiments, the same or similar elements will use the same or similar reference numerals.

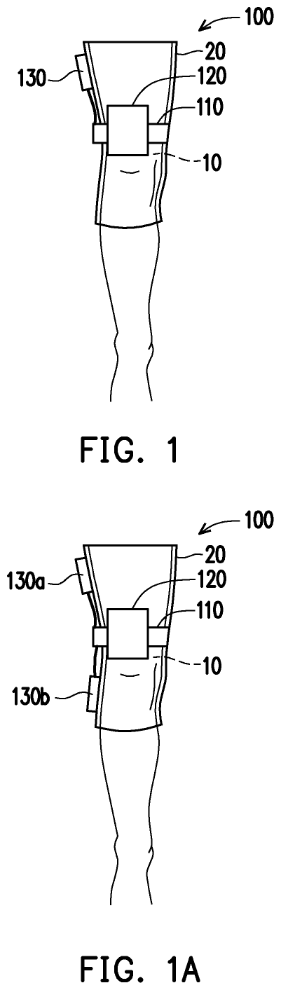

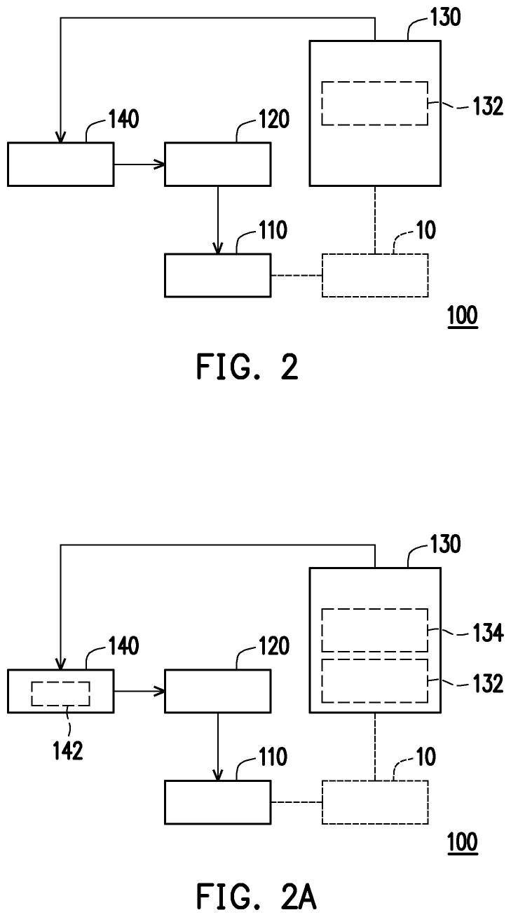

FIG. 1 is a schematic diagram of an automatic adjustable supporting equipment when in use according to an embodiment of the disclosure. FIG. 2 is a schematic block diagram of the automatic adjustable supporting equipment according to an embodiment of the disclosure. With reference to FIGS. 1 and 2 concurrently, in some embodiments, an automatic adjustable supporting equipment 100 may be worn on a limb 10 of a user to provide support and protection to the limb 10. In some embodiments, the automatic adjustable supporting equipment 100 may be worn on waist, four limbs, a joint or other suitable body parts of the user, that is, the limb 10 may include the waist, the four limbs, the joint and the other body parts. In this embodiment, the limb 10 may be a knee joint of the user as shown in FIG. 1, but the disclosure is not limited thereto.

In some embodiments, the automatic adjustable supporting equipment 100 may include a supporting band 110, an actuating mechanism 120, a detection module 130, and a controller 140. The supporting band 110 is configured to wrap around the limb 10 of the user. In some embodiments, the supporting band 110 may directly wrap around the limb 10 of the user, such as the waist or at least one side of the joint of the user, so as to provide the support and protection to the limb 10 through adjustments such as directly tightening or releasing the limb 10. In other embodiments, the automatic adjustable supporting equipment 100 may additionally include a protection device 20 with higher mechanical strength (for example, a joint protection device such as a knee pad, or an elbow pad). The protection device 20 may cover the limb 10 of the user to prevent the limb 10 from being injured by external forces such as collision, and the supporting band 110 may wrap around the protection device 20 to perform adjustments on the protection device 20 such as tightening or loosening.

In some embodiments, the actuating mechanism 120 may be assembled with the supporting band 110, and is configured to adjust pressure applied on the limb 10 by the supporting band 110. In the embodiment, the detection module 130 may be configured to detect a motion parameter of the limb 10 (for example, acceleration, or an angle, etc.). The detection module 130 may be, for example, disposed on the limb 10 or on at least one side of the limb 10. In the embodiment, the automatic adjustable supporting equipment 100 may include a single detection module 130, that is, the number of the detection module 130 may be one, but the embodiment is not limited thereto. In other embodiments, the automatic adjustable supporting equipment may also include two or more detection modules to detect the motion parameter of the limb 10 respectively or cooperatively.

In some embodiments, the controller 140 is coupled to the actuating mechanism 120 and the detection module 130 to determine an action state of the user according to the motion parameter detected by the detection module 130, and drive the actuating mechanism 120 to adjust the pressure applied by supporting band 110 on the limb 10 according to the action state. In some embodiments, the controller 140 may control the actuating mechanism 120 to adjust the pressure applied by the supporting band 110 to a preset pressure value according to the action state of the user.

In the embodiment, the detection module 130 may include an accelerometer 132, and the accelerometer 132 is configured to detect an acceleration value of the limb 10. The controller 140 is coupled to the accelerometer 132 to determine the action state of the user according to the acceleration value detected by the accelerometer 132, and drive the actuating mechanism 120 to adjust the pressure applied on the limb 10 by the supporting band 110 according to the action state. The controller 140 may be disposed in the detection module 130, but may also be installed in the actuating mechanism 120.

For example, when the acceleration value detected by the accelerometer 132 is substantially greater than or equal to a preset acceleration value, the controller 140 may then determine that the limb 10 is in a dynamic action state accordingly, and drive the actuating mechanism 120 to increase the pressure applied by the supporting band 110 to a preset dynamic pressure value accordingly. In the embodiment, the preset acceleration value may be approximately between 1G and 2G, and the preset dynamic pressure value may be approximately between 3 kg/cm² and 12 kg/cm². However, the above numerical range is only a description of an example. Any person with ordinary knowledge in the technical field should understand that the above numerical range differs for different body parts and conditions of different users, and the disclosure is not limited thereto. In contrast, the controller 140 determines that the limb 10 is a static action state, and drive the actuating mechanism 120 to reduce the pressure applied by the supporting band 110 to a preset static pressure value accordingly when the acceleration value detected by the accelerometer 132 is substantially less than the preset acceleration value for a duration, and the duration of the status where the acceleration value is less than the preset acceleration value is substantially longer than a preset time period. In the embodiment, the preset acceleration value may be between 0G and 0.1G, and the preset static pressure value may be between 1 kg/cm² and 3 kg/cm². However, the above numerical range is only a description of an example. Any person with ordinary knowledge in the technical field should understand that the above numerical range differs for the different body parts and the conditions of the different users, and the disclosure is not limited thereto. In some embodiments, the accelerometer 132 may be a three-axis accelerometer to detect the acceleration value of the limb 10 in X direction, Y direction, and Z direction.

FIG. 1A is a schematic diagram of an automatic adjustable supporting equipment of another embodiment of the disclosure. It should be noted that the automatic adjustable supporting equipment of this embodiment is similar to the automatic adjustable supporting equipment in FIG. 1. Therefore, the embodiment continues to use the component numerals and part of the content of the previous embodiment, in which the same reference numerals are used to represent the same or similar components, and description of the same technical content is omitted. Reference may be made to the foregoing embodiment for the description of the omitted part, which will not be repeated in the embodiment. A difference between the automatic adjustable supporting equipment of the embodiment and the automatic adjustable supporting equipment in FIG. 1 is described as follows.

With reference to FIG. 1A, in the embodiment, the automatic adjustable supporting equipment may include two detection modules 130a and 130b, and the two detection modules 130a and 130b may detect the motion parameter of the limb 10 respectively or cooperatively. For example, when the limb 10 is the joint of the user, the detection modules 130a and 130b may be respectively disposed on two opposite sides of the joint to detect the motion parameter such as the acceleration value and/or an angle formed between opposite ends of the joint. Furthermore, the sensor modules 130a and 130b may be respectively disposed at thigh and calf connected to the knee joint near the knee joint as shown in FIG. 1A when the limb 10 is the joint of the user, so as to respectively detect the motion parameter (for example, a parameter such as acceleration, or an angle formed between the thigh and the calf) of the thigh and the calf

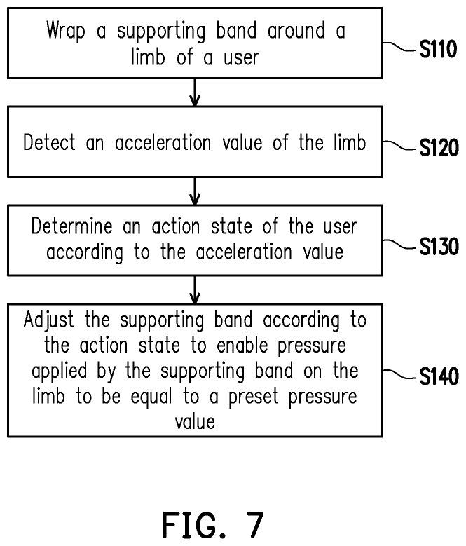

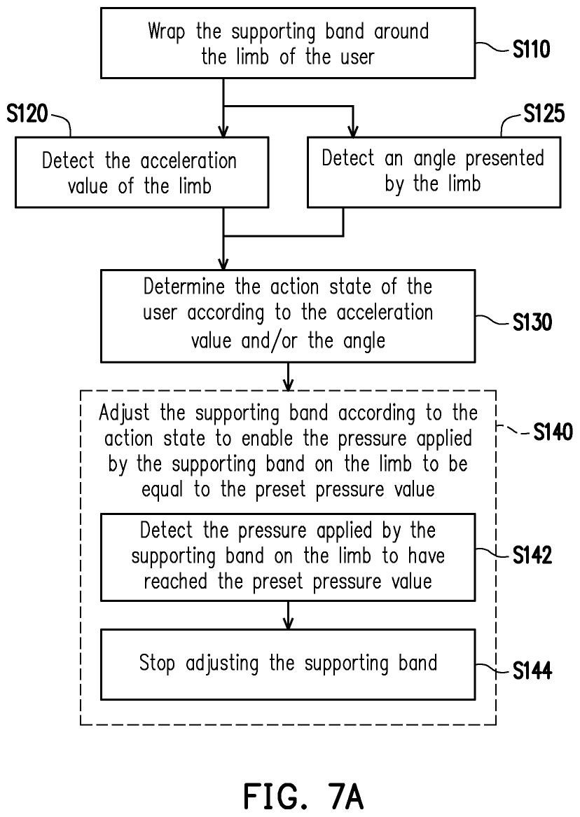

FIG. 7 is a schematic flowchart of a method for automatically adjusting the supporting equipment according to an embodiment of the disclosure. With reference to FIGS. 1, 2 and 7 concurrently, in the foregoing configuration, the method for automatically adjusting the supporting equipment may include the following steps. Firstly, the supporting equipment 100 is worn on the limb 10 of the user. For example, the supporting band 110 is wrapped around the limb 10 of the user (Step S110). Next, the motion parameter of the limb 10 is detected by the detection module 130. For example, the acceleration value of the limb 10 is detected (Step S120). In some embodiments, the accelerometer 132 may be, for example, disposed on the limb 10, on one side of the limb 10, or on two opposite sides of the limb 10. In the embodiment, the limb 10 may be, for example, the knee joint of the user, and the accelerometers 132 may be respectively disposed on, for example, the two opposite sides of the knee joint, so as to respectively detect the acceleration value of the limbs (such as the thigh and the calf) connected to the knee joint.

Then, Step S130 is executed to determine the action state (for example, the dynamic action state or the static action state) of the user according to the detected acceleration value. During a variety of action states of the user, the acceleration value detected by the accelerometer 132 may have a variety of different combinations of detection results. The controller 140 may match multiple different action states with their corresponding multiple different combinations of detection results, and then determine the action state (such as described in the previous example, but not limited thereto) of the user according to the different combinations of detection results detected by the detection module 130. Next, Step S140 is executed, in which the controller 140 adjusts the pressure applied on the limb 10 by the supporting band 110 according to the determined action state, for example, to enable the pressure to be approximately equal to the preset pressure value corresponding to the action state.

FIG. 2A is a schematic block diagram of an automatic adjustable supporting equipment according to another embodiment of the disclosure. It should be noted that the automatic adjustable supporting equipment of this embodiment is similar to the automatic adjustable supporting equipment in FIG. 2. Therefore, the embodiment continues to use the component numerals and part of the content of the previous embodiment, in which the same reference numerals are used to represent the same or similar components, and description of the same technical content is omitted. Reference may be made to the foregoing embodiment for the description of the omitted part, which will not be repeated in the embodiment. With reference to FIGS. 1 and 2A, a difference between the automatic adjustable supporting equipment of the embodiment and the automatic adjustable supporting equipment 100 in FIG. 1 is described as follows.

In some embodiments, the detection module 130 may further include a pressure sensor 134 coupled to the controller 140 and the supporting band 110 to detect the pressure applied on the limb 10 by the supporting band 110. In the embodiment, the controller 140 may be disposed in the detection module 130, in other embodiments, the controller may be disposed in the actuating mechanism. The pressure sensor 134 of the embodiment may be, for example, disposed on a surface of the supporting band 110 in contact with the limb 10. In this configuration, the controller 140 stops driving the actuating mechanism 120 to operate when the pressure detected by the pressure sensor 134 is approximately equal to the preset pressure value. For example, the controller 140 drives the actuating mechanism 120 to increase the pressure applied by the supporting band 110 when it determines that the limb 10 is in the dynamic action state. The controller 140 stops driving the actuating mechanism 120 operating when the pressure detected by the pressure sensor 134 reaches (actual value is greater than or equal to) the preset dynamic pressure value. That is, the controller 140 stops tightening the supporting band 110. In contrast, the controller 140 drives the actuating mechanism 120 to reduce the pressure applied by the supporting band 110 when it determines that the limb 10 is in the static action state. The controller 140 stops driving the actuating mechanism 120 to operate when the pressure detected by the pressure sensor 134 reaches (actual value is less than or equal to) the preset static pressure value. That is, the controller 140 stops releasing the supporting band 110.

In such a configuration, the automatic adjustable supporting equipment 100 of the disclosure may determine the action state of the user according to the motion parameter of the limb detected by the sensor, and drive the actuating mechanism 120 to adjust (increase or reduce) the pressure applied on the limb 10 by the supporting band 110 according to the action state. Therefore, the actuating mechanism 120 may increase the pressure (tightening) applied on the limb 10 by the supporting band 110, so as to increase supporting force and restraining force on the limb 10 when the user is in the dynamic action state (such as walking, running, falling, sitting to standing, or standing to sitting). The actuating mechanism 120 may reduce the pressure (releasing) applied on the limb 10 by the supporting band 110, so to improve comfort of the user when the user is in the static action state (such as sitting, lying, or standing).

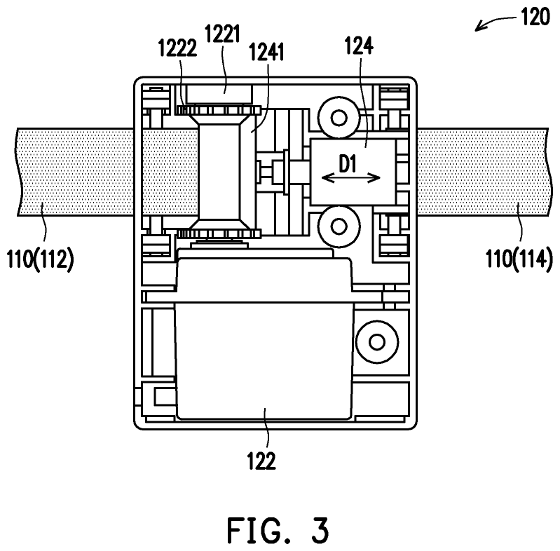

FIG. 3 is a schematic diagram of an actuating mechanism of the automatic adjustable supporting equipment according to an embodiment of the disclosure. In the embodiment, the actuating mechanism 120 includes a motor 122 and an electromagnetic valve 124. In some embodiments, the motor 122 may be connected to the supporting band 110. The motor 122 is configured to drive the supporting band 110, so as to adjust the pressure applied on the limb 10 by the supporting band 110. In the embodiment, the motor 122 may be, for example, a spindle motor, which may include an axis of rotation 1221. The motor 122 is configured to drive the axis of rotation 1221 to rotate. In the embodiment, at least one end of the supporting band 110 may be disposed on the axis of rotation 1221. Therefore, the end of the supporting band 110 may be driven to rotate through the axis of rotation 1221, so as to adjust the elasticity of the supporting band 110, thereby adjusting the pressure applied on the limb 10 by the supporting band 110. In the embodiment, the supporting band 110 may include a movable part 112 and a fixed part 114. An end of the movable part is disposed on the axis of rotation 1221 to allow the elasticity of the supporting band 110 to be adjusted with the rotation of the axis of rotation 1221, while the fixed part 114 maintains fixed.

In some embodiments, the electromagnetic valve 124 is coupled to the controller 140, so as to be controlled by the controller 140 to switch between an engaged position and a rotation position in a switch direction D1. In the embodiment, circumference of the axis of rotation 1221 may include multiple teeth 1222, and the electromagnetic valve 124 may include a stopping piece 1241 suitable for engagement with the teeth 1222. In this configuration, the controller 140 controls the electromagnetic valve 124 to switch to the engaged position as shown in FIG. 3 when the controller 140 wants to stop driving the actuating mechanism 120 to operate (for example, when the pressure detected by the pressure sensor 134 is approximately equal to the preset pressure value), enabling the stopping piece 1241 of the electromagnetic valve 124 to be engaged with the teeth 1222 of the axis of rotation 1221 of the motor 122, so as to block rotation of the motor 122 and stop driving the motor 122 to rotate. In this way, the end of the supporting band 110 stops being driven to roll and the elasticity of the supporting band 110 may be fixed.

Similarly, the controller 140 controls the electromagnetic valve 124 to move in the switch direction D1 (for example, move to the right as shown in FIG. 3) to the rotation position and drives the motor 122 to start rotating when the controller 140 wants to adjust the pressure applied on the limb 10 by the supporting band 110 (for example, when the controller 140 determines that the action state of the user has changed). At this time, the stopping piece 1241 of the electromagnetic valve 124 and the teeth 1222 of the axis of rotation 1221 of the motor 122 are disengaged, enabling the motor 122 to rotate freely. In this way, the end of the supporting band 110 starts to be driven to rotate and the elasticity of the supporting band 110 may be adjusted. The controller 140 may be disposed in the detection module, but may also be disposed in the actuating mechanism 120.

In this configuration, the automatic adjustable supporting equipment 100 of the disclosure only has to switch the electromagnetic valve 124 to the engaged position when the actuating mechanism 120 of the automatic adjustable supporting equipment 100 is to be stopped. This makes the motor 122 and the end of the supporting band 110 to be fixed at current position, thereby fixing current elasticity of the supporting band 110. Then, instead of continuously supplying power to the actuating mechanism 120, the elasticity of the supporting band 110 is maintained only by the mechanical engagement relationship between the stopping piece 1241 of the electromagnetic valve 124 and the axis of rotation 1221 of the motor 122. Therefore, the automatic adjustable supporting equipment 100 of the disclosure can not only automatically adjust the elasticity of the supporting band 110, but also reduce power consumption.

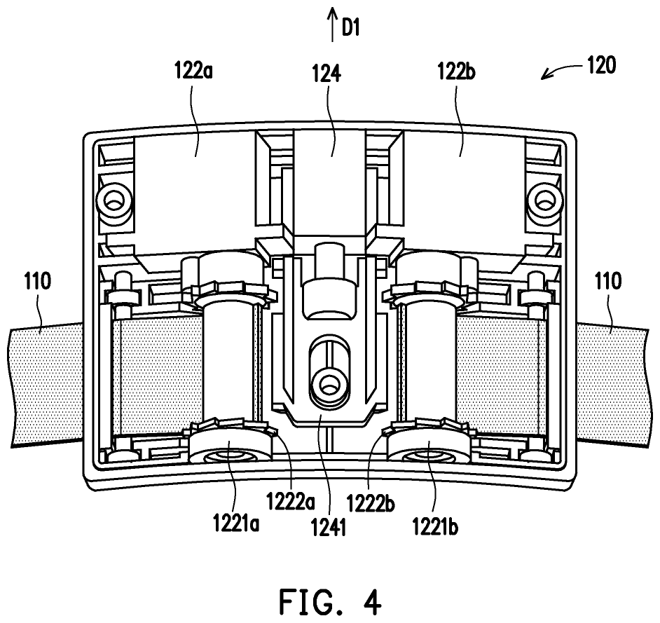

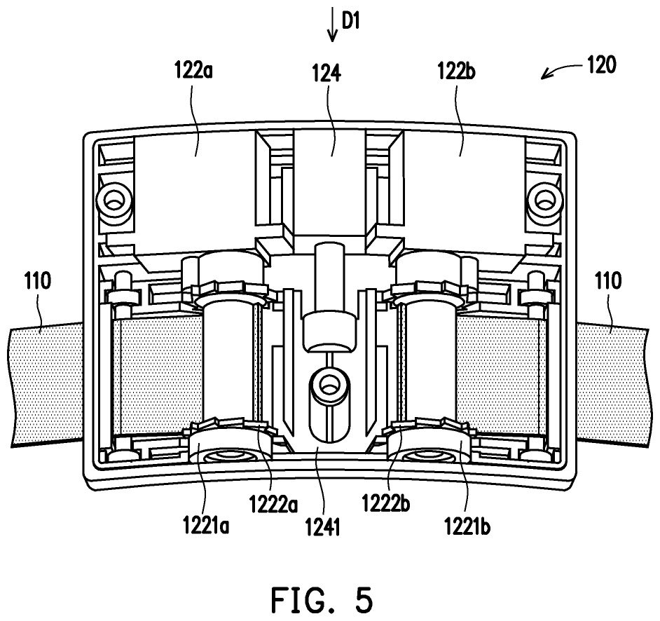

FIGS. 4 and 5 are schematic diagrams of the actuating mechanism of the automatic adjustable supporting equipment in two statuses according to another embodiment of the disclosure. It should be noted here that the actuating mechanism 120 of this embodiment is similar to the actuating mechanism 120 in FIG. 3. Therefore, the embodiment continues to use the component numerals and part of the content of the previous embodiment, in which the same reference numerals are used to represent the same or similar components, and description of the same technical content is omitted. Reference may be made to the foregoing embodiment for the description of the omitted part, which will not be repeated in the embodiment. With reference to FIGS. 4 and 5, a difference between the actuating mechanism 120 of the embodiment and the actuating mechanism 120 in FIG. 3 is described as follows.

In the embodiment, the actuating mechanism 120 may include multiple motors 122a and 122b, which are respectively assembled with two opposite ends of the supporting band 110, so as to drive the two ends (for example, to be in relative rotation) of the supporting band 110 to roll and adjust the pressure applied on the limb 10 by the supporting band 110. Therefore, the actuating mechanism 120 of the embodiment uses the two motors 122a and 122b to respectively drive the two opposite ends of the supporting band 110 to rotate in opposite directions concurrently. This may accelerates the efficiency of adjusting the elasticity of the supporting band 110. That is, a shorter period of time is required to adjust the supporting band 110 to a desired preset pressure value.

In the embodiment, the motors 122a and 122b respectively include axes of rotation 1221a and 1221b. Circumferences of the axes of rotation 1221a and 1221b may respectively include multiple teeth 1222a and 1222b. In some embodiments, rotation directions of the axes of rotation 1221a and 1221b of the motors 122a and 122b are opposite to each other, and the motors 122a and 122b are respectively assembled with the two opposite ends of the supporting band 110, so as to drive the two ends (for example, to rotate in the opposite direction to retract or release) of the supporting band 110 to roll. The electromagnetic valve 124 may be, for example, disposed between the two motors 122a and 122b. In this configuration, the controller 140 controls the electromagnetic valve 124 to switch to the rotation position shown in FIG. 4, and drive the motor 122 to start rotating when the controller 140 wants to adjust the pressure applied on the limb 10 by the supporting band 110 (for example, when the controller determines that the action state of the user has changed). At this time, the stopping piece 1241 of the electromagnetic valve 124 is disengaged from the teeth 1222 of the axis of rotation 1221 of the motor 122, enabling the motor 122 to rotate freely. In this way, the two ends of the supporting band 110 start to rotate relative to each other and the elasticity of the supporting band 110 may be adjusted.

In contrast, the controller 140 controls the electromagnetic valve 124 to switch to the engaged position as shown in FIG. 5 when the controller 140 wants to stop driving the actuating mechanism 120 to operate. For example, when the pressure detected by the pressure sensor 134 is approximately equal to the preset pressure value. This enables the stopping piece 1241 of the electromagnetic valve 124 to be respectively engaged with the teeth 1222a and 1222b of the axes of rotation 1221a and 1221b of the motors 122a and 122b, so as to block rotation of the motors 122a and 122b, and the controller 140 stops driving the motor 122 to rotate. At this time, the stopping piece 1241 is engaged with the teeth 1222a and 1222b of the axes of rotation 1221a and 1221b, which enables positioning of the motor 122. In this way, the two opposite ends of the supporting band 110 stop retracting and the elasticity of the supporting band 110 may be fixed. The controller 140 may be disposed in the detection module, but in other embodiments, the controller may be disposed in the actuating mechanism 120.

In such a configuration, the automatic adjustable supporting equipment of the embodiment uses the two motors 122a and 122b to respectively drive the two opposite ends of the supporting band 110 to roll in the opposite directions concurrently, therefore the supporting band 110 may be adjusted to the desired preset pressure value more quickly. In addition, the actuating mechanism 120 only has to control one electromagnetic valve 124 to switch to the engaged position when the actuating mechanism 120 wants to stop driving the actuating mechanism 120 to operate. This allows engagement with the two motors 122a and 122b concurrently, and stops driving of the two opposite ends of the supporting band 110 to roll, and the two opposite ends of the supporting band 110 are fixed at current position, thereby fixing the current elasticity of the supporting band 110. There is no need to continuously supply power to the actuating mechanism 120 after the electromagnetic valve 124 is in the engaged position. At this time, the elasticity of the supporting band 110 is only maintained by the mechanical engagement relationship between the stopping piece 1241 of the electromagnetic valve 124 and the axes of rotation 1221a and 1221b of the motor 122a and 122b. Therefore, the automatic adjustable supporting equipment of the embodiment can not only improve efficiency of automatically adjusting the elasticity of the supporting band 110, but also reduce the power consumption.

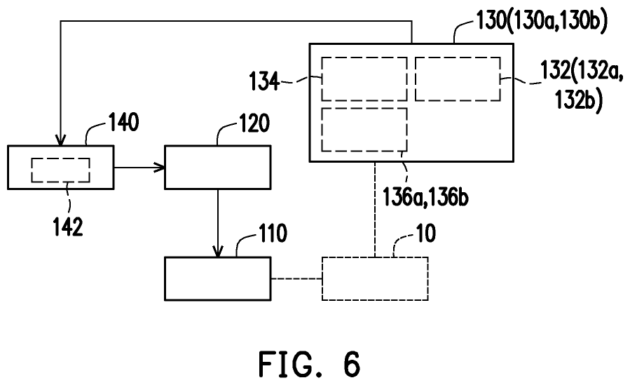

FIG. 6 is a schematic block diagram of the automatic adjustable supporting equipment according to another embodiment of the disclosure. FIG. 7A is a schematic flowchart of a method for automatically adjusting the supporting equipment according to another embodiment of the disclosure. With reference to FIGS. 6 and 7A concurrently, in some embodiments, the automatic adjustable supporting equipment 100 may be applied to protection of the joint, that is, the limb 10 includes the joint of the user and the two limb parts 11 and 12 connected to the joint. In the embodiment, the automatic adjustable supporting equipment 100 may include multiple sensor modules 130a and 130b, which are respectively disposed on the two opposite sides of the joint to detect the motion parameters of the two sides of the joint. For example, if the automatic adjustable supporting equipment 100 is configured to protect the knee joint, the sensor modules 130a and 130b may be respectively disposed on the two limbs 11 and 12 connected to the knee joint, as shown in FIG. 1, namely the thigh and the calf near the knee joint, so as to respectively detect the motion parameters (such as the acceleration and the angle formed between the thigh and the calf) of the thigh and calf. In some embodiments, the accelerometer 132 may include multiple accelerometers 132a and 132b, which are respectively disposed on the limbs 11 and 12 that are connected to the joint. In some embodiments, the automatic adjustable supporting equipment 100 may further include multiple angle sensors 136a and 136b, which are coupled to the controller 140 and respectively disposed on the limbs 11 and 12 that are connected to the joint, so as to detect the angle of the joint. In the embodiment, the angle sensors 136a and 136b may be gyroscopes, but the embodiment is not limited thereto.

In such a configuration, the method for automatically adjusting the supporting equipment may include the following steps. Firstly, the supporting equipment 100 is worn on the limb 10 of the user. For example, the supporting band 110 is wrapped around the limb 10 of the user (the Step S110). Next, the motion parameter of the limb is detected by the detection module 130. For example, the acceleration value of the limb 10 is detected (the Step S120) and/or the angle presented by the limb 10 is detected (Step S125). In the embodiment, the accelerometers 132a and 132b may be, for example, respectively disposed on the two opposite sides of the knee joint, so as to respectively detect the acceleration value of the limbs 11 and 12 (for example, the thigh and the calf) connected to the knee joint. The angle sensors 136a and 136b then may be respectively disposed on the two opposite sides of the joint, that is, respectively disposed on the limbs 11 and 12 connected to the joint, so as to detect angle of the knee joint, that is, the angle formed between the limbs 11 and 12 (such as the thigh and the calf) connected to the knee joint.

Then, Step S130 is executed to determine the action state (for example, the dynamic action state or the static action state) of the user according to the detected acceleration value and/or the angle. During the variety of action states of the user, the acceleration value detected by the accelerometers 132a and 132b, and the angle detected by the angle sensors 136a and 136b may have a variety of different combinations of detection results. The controller 140 may match the multiple different action states with their corresponding multiple different combinations of detection results, and then determine the action state of the user according to the different combinations of detection results detected by the detection module 130. Several action states and their corresponding combinations of detection results are listed as examples in the following, but the disclosure is not limited thereto. Then, the Step S140 is executed, in which the controller 140 adjusts the pressure applied on the limb 10 by the supporting band 110 according to the determined action state, for example, to enable the pressure to be approximately equal to the preset pressure value corresponding to the action state. The method that enables the pressure to be approximately equal to the preset pressure value (the Step S140) corresponding to the action state may include the following substeps. For example, the pressure sensor 134 is used to detect the pressure applied on the limb 10 by the supporting band 110. The controller 140 stops adjusting the pressure applied on the limb 10 by the supporting band 110 (Step S144), that is, the controller 140 fixes the elasticity of the current supporting band 110 when the pressure applied on the limb 10 by the supporting band 110 is detected by the pressure sensor 134 to be approximately equal to the preset pressure value (Step S142).

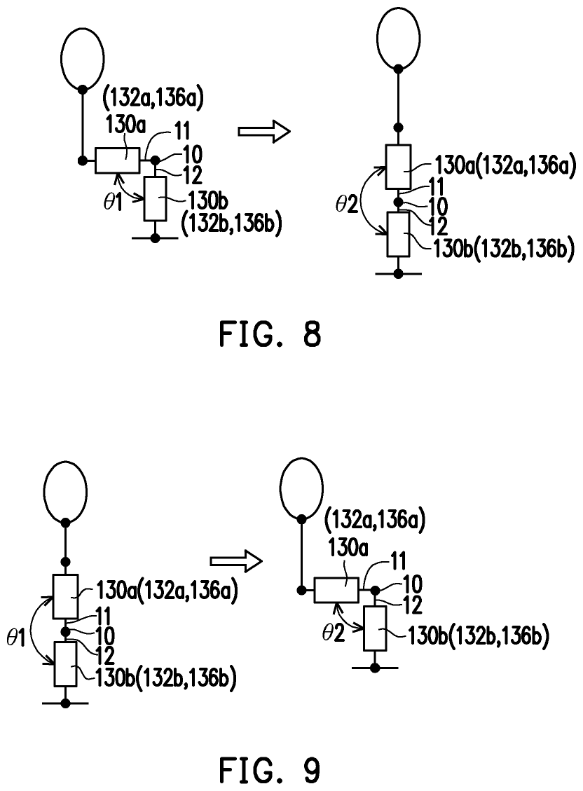

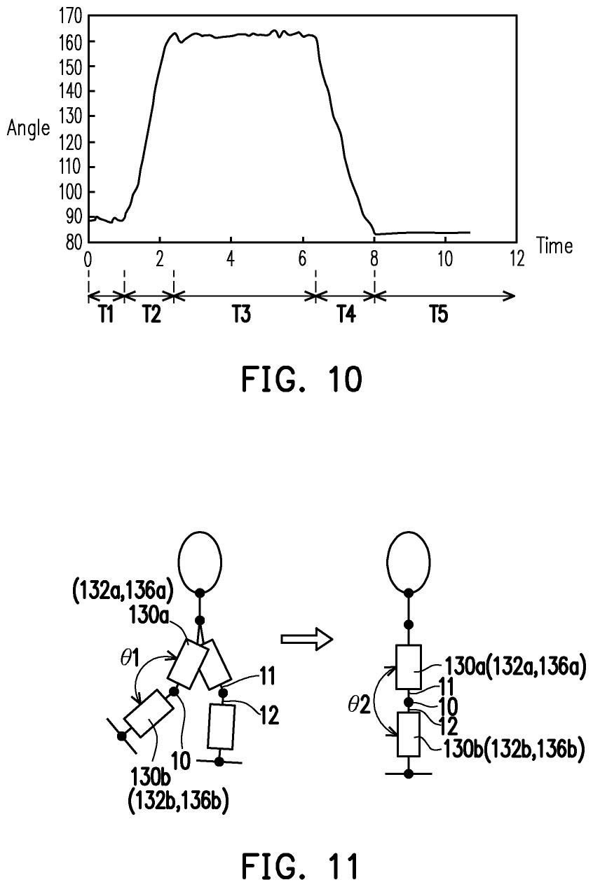

FIGS. 8 and 9 are schematic diagrams of the automatic adjustable supporting equipment when used in different action states according to an embodiment of the disclosure. FIG. 10 is a schematic curve diagram of angles detected by an angle sensor in different action states according to an embodiment of the disclosure. With reference to FIGS. 8 and 10 first, in an embodiment of the disclosure, the angle of the knee joint (the limb 10) of the user will increase from approximately 90 degrees to close to 180 degrees (from angle θ1 to angle θ2) when the user changes the action state from sitting to standing, as shown in FIG. 8. Since the thigh and the calf of the user are both in motion, their acceleration value will also increase. Therefore, the controller 140 determines that the limb 10 is in the action state of sitting-to-standing, which belongs to the dynamic action state when the acceleration value detected by the accelerometers 132a and 132b is substantially greater than or equal to the preset acceleration value and the angle detected by the angle sensors 136a and 136b increases. This drives the actuating mechanism 120 to adjust the supporting band 110 to increase the pressure applied on the limb 10 by the supporting band 110 accordingly, for example, to increase the pressure to the preset dynamic pressure value.

It should be noted that the time-dependent curve of the joint angle as detected by the angle sensors 136a and 136b is shown in FIG. 10. During a time period T1, the user is in the action state of sitting, therefore the angle of the knee joint is approximately maintained at 90 degrees. During a time period T2, the user changes from the action state of sitting to standing, therefore the angle of the knee joint increases from approximately 90 degrees to close to 180 degrees. During a time period T3, the user maintains the action state of standing, while during a time period of T4, the user changes from the action state of standing to sitting. Therefore, the controller 140 of the automatic adjustable supporting equipment 100 of the embodiment may determine the action state of the user from the curve of the angle. In some embodiments, the controller 140 may also determine the action state of the user according to the angle detected by the angle sensors 136a and 136b. In addition, because limb posture, angle, and momentum of the user may be slightly different every time, and the angle and the momentum presented by different users in the same posture will also be different, values such as the angle, the acceleration value, and the pressure mentioned in the disclosure are all just examples. Terms such as “substantially”, “approximately”, “left”, or “right” and other terms represent an allowance of at least ±15% error. The controller 140 may be disposed in the detection module, but in other embodiments, the controller may be disposed in the actuating mechanism.

With reference to FIGS. 9 and 10 again, in an embodiment of the disclosure, the angle of the knee joint (the limb 10) of the user will reduce from approximately 180 degrees to close to 90 degrees (such as during the time period T4 in FIG. 10) when the user changes the action state from standing to sitting, as shown in FIG. 9. In addition, since the thigh and the calf of the user are close to the static action state after sitting down, the acceleration value of the thigh and the calf will be greatly reduced and maintained at a low level for a period of time. Therefore, the controller 140 determines that the limb 10 is in the action state of sitting, which belongs to the static action state when the angle detected by the angle sensors 136a and 136b decreases, the acceleration value detected by the accelerometers 132a and 132b is substantially less than the preset acceleration value for a duration, and the duration is substantially greater than or equal to the preset time period (for example, approximately 10 seconds). This drives the actuating mechanism 120 to adjust the supporting band 110 to reduce the pressure applied on the limb 10 by the supporting band 110 accordingly, for example, to reduce the pressure to the preset static pressure value. The controller 140 may be disposed in the detection module, but in other embodiments, the controller may be disposed in the actuating mechanism 120.

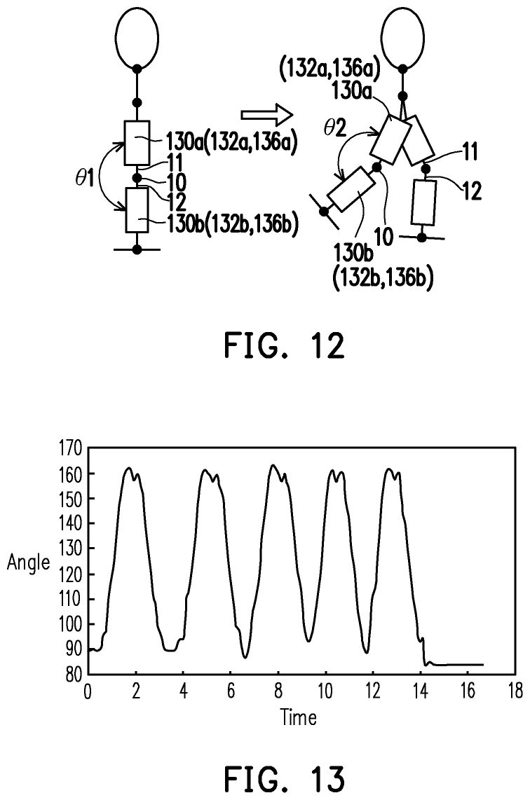

FIGS. 11 and 12 are schematic diagrams of the automatic adjustable supporting equipment when used in different action states according to an embodiment of the disclosure. FIG. 13 is a schematic curve diagram of the angles detected by an angle sensor in different action states according to an embodiment of the disclosure. With reference to FIG. 11 first, in an embodiment of the disclosure, the angle presented by the knee joint of the user increases from approximately 135 degrees to close to 180 degrees (from angle θ1 to angle θ2) when the user changes the action state from walking to standing, as shown in FIG. 11. In addition, since the thigh and the calf of the user are close to the static action state while standing, the acceleration value of the thigh and the calf will be greatly reduced and maintained at a low level for a period of time. Therefore, the controller 140 determines that the limb 10 is in the action state of standing, which belongs to the static action state when the angle detected by the angle sensors 136a and 136b gradually increases to close to 180 degrees, the acceleration value detected by the accelerometers 132a and 132b is substantially less than the preset acceleration value for a duration, and the duration is substantially greater than or equal to the preset time period (for example, approximately 10 seconds). The controller 140 drives the actuating mechanism 120 to adjust the supporting band 110 accordingly to reduce the pressure applied on the limb 10 by the supporting band 110, for example, to reduce the pressure to the preset static pressure value. The controller 140 may be disposed in the detection module, but in other embodiments, the controller may be disposed in the actuating mechanism 120.

With reference to FIGS. 12 and 13, in an embodiment of the disclosure, the angle presented by the knee joint is changing continuously as the user starts walking when the user changes from the action state of standing to walking, as shown in FIGS. 12 and 13. Because the thigh and the calf of the user are in motion, their acceleration value increases and there is a larger acceleration value. Therefore, the controller 140 determines that the limb 10 is the action state of walking, which belongs to the dynamic action state when the acceleration value detected by the accelerometers 132a and 132b is increasing continuously, and the angle detected by the angle sensors 136a and 136b is also changing continuously. The controller 140 drives the actuating mechanism 120 to adjust the supporting band 110 accordingly to increase the pressured applied on the limb 10 by the supporting band 110, for example, to increase the pressure to the preset dynamic pressure value. The controller 140 may be disposed in the detection module, but in other embodiments, the controller may be disposed in the actuating mechanism 120.

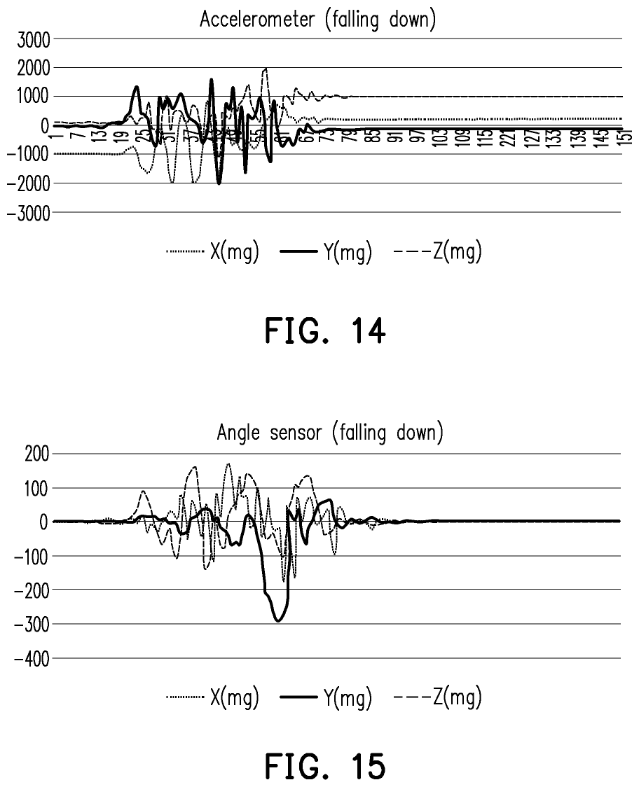

FIG. 14 is a schematic curve diagram of accelerations detected by an accelerometer in another action state according to an embodiment of the disclosure. FIG. 15 is a schematic curve diagram of the angles detected by the angle sensor in another action state according to an embodiment of the disclosure. The user may have a variety of different action states, and the variety of action states have their corresponding different action trajectories. For example, the action states may include walking, running, falling down, squatting, kneeling, lying down, etc., in which the action trajectories of some of the action states are more complicated. FIGS. 14 and 15 show the values read by the accelerometer and the angle sensor during the action state of “falling down”, in which changes of the values in all directions are more complicated, and the acceleration and the angle of each fall may also be totally different. Therefore, in some embodiments, the controller 140 further includes at least one action identification model 142 (as shown in FIG. 2A), and the action identification model 142 of the controller 140 may determine the action state of the user according to the detection results of the accelerometers 132a and 132b, and the angle sensors 136a and 136b. In the embodiment, the action identification model 142 may, for example, use Python to build a neural model training database, input the detection results of the accelerometers 132a and 132b, and the angle sensors 136a and 136b, and enables the action identification model 142 to perform feature analysis and capturing on the detection results, so as to identify (determine) the action state of the user through machine learning and identification algorithm. Evidently, the embodiment is only for illustration, and the disclosure is not limited thereto.

After the controller 140 has determined the action state of the user, the controller 140 drives the actuating mechanism 120 to adjust (increase or reduce) the pressure applied by the supporting band 110 to the preset pressure value, for example, increase the pressure to the preset dynamic pressure value or reduce the pressure to the preset static pressure value. When the pressure applied on the limb 10 by the supporting band 110 detected by the pressure sensor 134 is approximately equal to the preset pressure value (the Step S142), the Step S144 is executed, in which the controller 140 stops adjusting the pressure applied on the limb 10 by the supporting band 110, which is also to fix the current elasticity of the supporting band 110. The controller 140 may be disposed in the detection module, but in other embodiments, the controller may be disposed in the actuating mechanism.

Based on the above, it can be seen that the embodiments of the disclosure provide various advantages. However, it should be understood that not all advantages are discussed herein, and other embodiments may provide different advantages, and not all embodiments require a specific advantage.

In summary, the automatic adjustable supporting equipment of the disclosure may determine the action state of the user according to the motion parameters of the limb detected by the sensor, and drive the actuating mechanism to adjust (increase or reduce) the pressure applied on the limb by the supporting band according to the action state. Therefore, the actuating mechanism may increase the pressure (tightening) applied on the limb by the supporting band to increase supporting force and restraining force on the limb when the user is in the dynamic action state. On the other hand, the actuating mechanism may reduce the pressure (releasing) applied on the limb by the supporting band to improve the comfort of the user when the user is in the static action state.

In addition, the automatic adjustable supporting equipment of the disclosure only needs to control the electromagnetic valve to switch to the engaged position when the automatic adjustable supporting equipment wants to stop the actuating mechanism. This enables the motor and the end of the supporting band to be fixed at the current position, thereby fixing the current elasticity of the supporting band. Then, instead of continuously supplying power to the actuating mechanism, the elasticity of the supporting band is only maintained by the engaging relationship between the stopping piece of the electromagnetic valve and the axis of rotation of the motor. Therefore, the automatic adjustable supporting equipment disclosed in the disclosure can not only automatically adjust the elasticity of the supporting band, but also reduce the power consumption.

Although the disclosure has been disclosed with the foregoing exemplary embodiments, it is not intended to limit the disclosure. Any person skilled in the art can make various changes and modifications within the spirit and scope of the disclosure. Accordingly, the scope of the disclosure is defined by the claims appended hereto and their equivalents.

What is claimed is:

1. An automatic adjustable supporting equipment, comprising:
a supporting band, suitable for wrapping around a limb of a user;
an actuating mechanism, assembled with the supporting band, and is configured to adjust a pressure applied on the limb by the supporting band;
an accelerometer, configured to detect an acceleration value; and
a controller, coupled to the actuating mechanism and the accelerometer, and is configured to drive the actuating mechanism to adjust the pressure to a preset pressure value according to the acceleration value.

2. The automatic adjustable supporting equipment according to claim 1, further comprising:
a pressure sensor, coupled to the controller and the supporting band, and is configured to detect the pressure.

3. The automatic adjustable supporting equipment according to claim 2, wherein the controller is configured to further determine an action state of the user according to the acceleration value, and the controller stops driving the actuating mechanism to operate when the pressure detected by the pressure sensor reaches the preset pressure value corresponding to the action state.

4. The automatic adjustable supporting equipment according to claim 1, wherein the actuating mechanism comprises:
a motor, configured to drive the supporting band to adjust the pressure applied on the limb by the supporting band; and
an electromagnetic valve, configured to be controlled by the controller to be capable of switching between an engaged position and a rotation position.

5. The automatic adjustable supporting equipment according to claim 4, wherein the electromagnetic valve comprises a stopping piece, the motor comprises an axis of rotation, the stopping piece of the electromagnetic valve is engaged with the axis of rotation of the motor to block rotation of the motor when the electromagnetic valve is in the engaged position, and the stopping piece of the electromagnetic valve is disengaged from the axis of rotation of the motor to enable the motor to rotate freely when the electromagnetic valve is in the rotation position.

6. The automatic adjustable supporting equipment according to claim 4, wherein the actuating mechanism comprises a plurality of motors that are respectively assembled with two opposite ends of the supporting band, and are configured to drive the two opposite ends to roll to adjust the pressure applied on the limb by the supporting band.

7. The automatic adjustable supporting equipment according to claim 6, wherein rotation directions of the plurality of motors are opposite to each other.

8. The automatic adjustable supporting equipment according to claim 1, wherein the controller drives the actuating mechanism to adjust the supporting band to increase the pressure applied on the limb by the supporting band to a preset dynamic pressure value when the acceleration value detected by the accelerometer is greater than or equal to a preset acceleration value.

9. The automatic adjustable supporting equipment according to claim 1, wherein the controller drives the actuating mechanism to adjust the supporting band to reduce the pressure applied on the limb by the supporting band to a preset static pressure value when the acceleration value detected by the accelerometer is less than a preset acceleration value for a duration and the duration is greater than or equal to a preset time period.

10. The automatic adjustable supporting equipment according to claim 1, comprising a plurality of accelerometers.

11. The automatic adjustable supporting equipment according to claim 10, further comprising:
a plurality of angle sensors, coupled to the controller, and configured to detect an angle of the limb.

12. The automatic adjustable supporting equipment according to claim 11, wherein the controller is configured to drive the actuating mechanism to adjust the supporting band and the pressure applied on the limb by the supporting band according to the acceleration value and the angle.

13. The automatic adjustable supporting equipment according to claim 11, wherein the angle sensors comprise a gyroscope.

14. The automatic adjustable supporting equipment according to claim 11, wherein the controller drives the actuating mechanism to adjust the supporting band and increase the pressure applied on the limb by the supporting band when the acceleration value is greater than or equal to a preset acceleration value and the angle is increased.

15. The automatic adjustable supporting equipment according to claim 11, wherein the controller drives the actuating mechanism to adjust the supporting band and reduce the pressure applied on the limb by the supporting band when the angle is decreased, and the acceleration value is less than a preset acceleration value for a duration, and the duration is greater than or equal to a preset time period.

16. The automatic adjustable supporting equipment according to claim 11, wherein the controller drives the actuating mechanism to adjust the supporting band and reduce the pressure applied on the limb by the supporting band when the angle is increased to greater than or equal to 180 degrees, the acceleration value is less than a preset acceleration value for a duration, and the duration is greater than or equal to a preset time period.

17. The automatic adjustable supporting equipment according to claim 11, wherein the controller drives the actuating mechanism to adjust the supporting band and increase the pressure applied on the limb by the supporting band when the angle is changing continuously and the acceleration value is changing continuously.

18. The automatic adjustable supporting equipment according to claim 1, wherein the controller determines an action state of the user by using at least one action identification model.

19. An automatic adjustable supporting equipment, comprising:
a supporting band, suitable for wrapping around a limb of a user;
an actuating mechanism, comprising:
a motor, assembled with the supporting band, and is configured to drive the supporting band to adjust a pressure applied on the limb by the supporting band; and
an electromagnetic valve, configured to be capable of switching between an engaged position and a rotation position, wherein the electromagnetic valve comprises a stopping piece; and
a controller, coupled to the actuating mechanism, and configured to control the electromagnetic valve to switch to the engaged position or the rotation position,
wherein the stopping piece of the electromagnetic valve is engaged with an axis of rotation of the motor to block rotation of the motor when the electromagnetic valve is in the engaged position, and the stopping piece of the electromagnetic valve is disengaged from the axis of rotation to enable the motor to rotate freely when the electromagnetic valve is in the rotation position.

20. The automatic adjustable supporting equipment according to claim 19, wherein actuating mechanism comprises a plurality of motors that are respectively assembled with two opposite ends of the supporting band, and the motors are configured to drive the two opposite ends to roll and adjust the pressure applied on the limb by the supporting band.

21. The automatic adjustable supporting equipment according to claim 19, further comprising:
an accelerometer, configured to detect an acceleration value, and coupled to the controller,
wherein the controller is further configured to drive the actuating mechanism to adjust the supporting band according to the acceleration value, so as to adjust the pressure applied on the limb by the supporting band to reach a preset pressure value.

22. The automatic adjustable supporting equipment according to claim 19, further comprising:
a pressure sensor, coupled to the controller and the supporting band, and configured to detect the pressure applied on the limb by the supporting band.

23. The automatic adjustable supporting equipment according to claim 22, wherein the controller stops driving the actuating mechanism to operate when the pressure detected by the pressure sensor reaches a preset pressure value.

24. The automatic adjustable supporting equipment according to claim 19, comprising a plurality of accelerometers.

25. The automatic adjustable supporting equipment according to claim 24, further comprising:
a plurality of angle sensors, coupled to the controller and respectively disposed on the supporting band to detect an angle of the limb.

26. The automatic adjustable supporting equipment according to claim 25, wherein the controller is configured to drive the actuating mechanism to adjust the supporting band according to the angle, so as to adjust the pressure to be equal to a preset pressure value.

27. The automatic adjustable supporting equipment according to claim 19, wherein the controller determines an action state of the user by using at least one action identification model.

28. A method for automatically adjusting a supporting equipment, comprising:
using a supporting band to wrap around a limb of a user;
detecting an acceleration value of the limb by an accelerometer; and
determining an action state of the user by a controller according to the acceleration value, and adjusting the supporting band accordingly to adjust a pressure applied on the limb by the supporting band to be equal to a preset pressure value, wherein the preset pressure value corresponds to the action state.

29. The method for automatically adjusting the supporting equipment according to claim 28, wherein adjustment of the supporting band accordingly by the controller and adjust the pressure applied on the limb by the supporting band to be equal to the preset pressure value comprises:
detecting the pressure applied on the limb by the supporting band by a pressure sensor; and
stopping adjusting the supporting band by the controller when the pressure detected by the pressure sensor is equal to the preset pressure value.

30. The method for automatically adjusting the supporting equipment according to claim 28, further comprising:
detecting an angle presented by the limb by an angle sensor, 6o1 wherein determination of the action state of the user by the controller according to the acceleration value comprises:
determining the action state of the user by the controller according to the acceleration value and the angle.

31. The method for automatically adjusting the supporting equipment according to claim 30, wherein the controller determines that the limb is in a dynamic action state, and adjusts the supporting band to increase the pressure applied on the limb by the supporting band to a preset dynamic pressure value when the acceleration value is substantially greater than or equal to a preset acceleration value and the angle is increased.

32. The method for automatically adjusting the supporting equipment according to claim 30, wherein the controller determines that the limb is in a static action state, and adjusts the supporting band to reduce the pressure applied on the limb by the supporting band to a preset static pressure value when the angle is decreased, the acceleration value is less than a preset acceleration value for a duration, and the duration is greater than or equal to a preset time period.

33. The method for automatically adjusting the supporting equipment according to claim 30, wherein the controller determines that the limb is in a static action state, and adjusts the supporting band to reduce the pressure applied on the limb by the supporting band to a preset static pressure value when the angle is increased to be equal to 180 degrees, the acceleration value is less than a preset acceleration value for a duration, and the duration is substantially greater than or equal to a preset time period.

34. The method for automatically adjusting the supporting equipment according to claim 30, wherein the controller determines that the limb is in a dynamic action state, and adjusts the supporting band to increase the pressure applied on the limb by the supporting band to a preset dynamic pressure value when the angle is changing continuously, the acceleration value is changing continuously.

35. The method for automatically adjusting the supporting equipment according to claim 28, wherein determination of the action state of the user by the controller according to the acceleration value comprises:
determining the action state of the user by using at least one action identification model by the controller.
